(19) United States
(12) Patent Application Publication
(10) Pub. No.: US 2013/0085548 A1
(43) Pub. Date: Apr. 4, 2013

(54) METHOD OF TREATING CHRONIC PAIN IN A PATIENT USING NEUROMODULATION

(71) Applicant: Eugene Y. Mironer, Spartanburg, SC (US)

(72) Inventor: Eugene Y. Mironer, Spartanburg, SC (US)

(21) Appl. No.: 13/633,996

(22) Filed: Oct. 3, 2012

Related U.S. Application Data
(60) Provisional application No. 61/542,554, filed on Oct. 3, 2011.

(51) Int. Cl. A61N 1/36; A61N 1/05

(52) U.S. Cl. 607/46; 607/117

(57) ABSTRACT

Some representative embodiments are directed to treating chronic pain in a patient. A first stimulation lead is implanted in the patient with electrodes in the epidural space of the patient. A second stimulation lead is implanted with electrodes in subcutaneous tissue in an area of back or torso pain of the patient. The electrodes of the second stimulation lead are disposed in a configuration that is substantially perpendicular to an axis defined by the spine of the patient. Electrical pulses are generated by an implantable pulse generator for application to tissue of the patient. The electrical pulses are applied to the tissue of the patient using electrodes of the first stimulation lead and electrodes of the second stimulation lead. Active electrodes on the first stimulation lead are set to a first polarity and active electrodes on the second stimulation lead are set to a second polarity that is opposite to the first polarity.

### RELATED APPLICATIONS

This application claims priority to U.S. Provisional Application Ser. No. 61/542,554, filed Oct. 3, 2011, entitled “METHOD OF TREATING CHRONIC PAIN IN A PATIENT USING NEUROMODULATION,” which is incorporated herein by reference.

### BACKGROUND

Neurostimulation systems are devices that generate electrical pulses and deliver the pulses to nerve tissue of a patient to treat a variety of disorders. Spinal cord stimulation (SCS) is the most common type of neurostimulation within the broader field of neuromodulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

Peripheral nerve field stimulation (PNFS) is another form of neuromodulation. The basic devices employed for PNFS are similar to the devices employed for SCS including pulse generators and stimulation leads. In PNFS, the stimulation leads are placed in subcutaneous tissue (hypodermis) in the area in which the patient experiences pain. Electrical stimulation is applied to nerve fibers in the painful area. PNFS has been suggested as a therapy for a variety of conditions such as migraine, occipital neuralgia, trigeminal neuralgia, lower back pain, chronic abdominal pain, chronic pain in the extremities, and other conditions.

### SUMMARY

Some representative embodiments are directed to treating chronic pain in a patient. A first stimulation lead is implanted in the patient with electrodes in the epidural space of the patient. A second stimulation lead is implanted with electrodes in subcutaneous tissue in an area of back pain of the patient. The electrodes of the second stimulation lead are disposed in a configuration that is substantially perpendicular to an axis defined by the spine of the patient. Electrical pulses are generated by an implantable pulse generator for application to tissue of the patient. The electrical pulses are applied to the tissue of the patient using electrodes of the first stimulation lead and electrodes of the second stimulation lead. Active electrodes on the first stimulation lead are set to a first polarity and active electrodes on the second stimulation lead are set to a second polarity that is opposite to the first polarity.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiments disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 depicts a stimulation system according to one representative embodiment.

FIGS. 2A-2C respectively depict stimulation portions for inclusion at the distal end of a lead according to some representative embodiments.

FIG. 3 depicts a fluoroscopy image of an epidural lead and an PNFS lead implanted to treat chronic pain in a patient according to one representative embodiment.

FIG. 4 depicts a graphical representation of the implant of epidural and PNFS leads according to one representative embodiment.

FIGS. 5A-5F depict respective mechanisms for retaining a PNFS lead at a desired location.

FIG. 6 depicts a silicone anchor implant device for securing a stimulation lead within a patient between the epidural space and the subcutaneous pocket created for the implantable pulse generator.

### DETAILED DESCRIPTION

Representative embodiments are directed to methods of treating chronic pain in a patient and, in specific embodiments, chronic pain involving axial back pain or intractable back pain. Some representative embodiments provide one or more stimulation leads within the epidural space of the patient. Also, one or more other leads are implanted with the electrodes in subcutaneous tissue. The electrodes of these leads are oriented in a manner that is substantially perpendicular to the spinal axis of the patient. Also, the electrodes of the subcutaneous leads are placed in the area of the worst back pain. The patient receives stimulation from the epidural leads and PNFS from the subcutaneous leads. Also, in preferred embodiments, the stimulation is applied such that “cross-talk” occurs between electrodes of the epidural leads and electrodes of the subcutaneous leads. For example, one or more electrodes of the subcutaneous leads may be set as cathodes while simultaneously one or more electrodes of the epidural leads may be set as anodes (or vice versa). Using these implant locations and techniques, stimulation coverage of areas of chronic back pain may be obtained in a manner believed to be more thorough and consistent than other known methods.

FIG. 1 depicts stimulation system 100 that generates electrical pulses for application to tissue of a patient according to one embodiment. System 100 includes implantable pulse generator 150 that is adapted to generate electrical pulses for application to tissue of a patient. Implantable pulse generator 150 typically comprises a metallic housing that encloses controller 151, pulse generating circuitry 152, charging coil 153, battery 154, far-field and/or near field communication circuitry 155, battery charging circuitry 156, switching circuitry 157, etc. of the device. Controller 151 typically includes a microcontroller or other suitable processor for controlling the various other components of the device. Software code is typically stored in memory of the pulse generator 150 for execution by the microcontroller or processor to control the various components of the device.

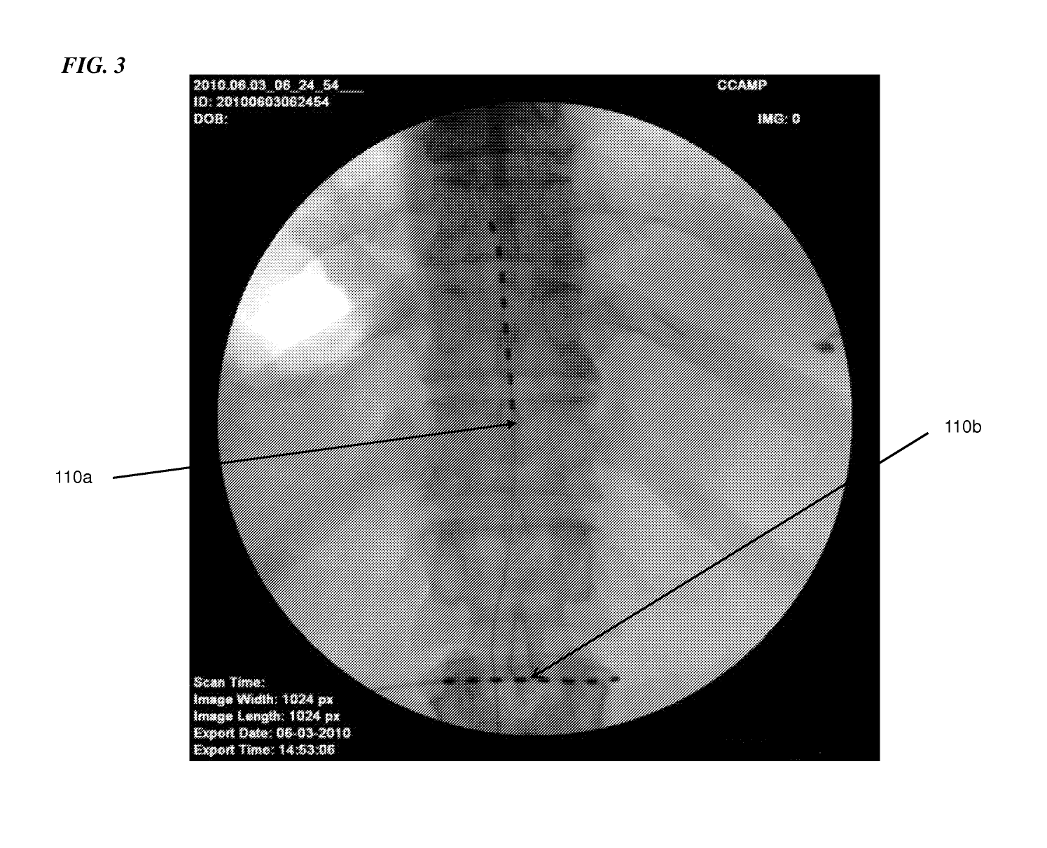

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry 157. The switching circuit connects to output wires, traces, lines, or the like (not shown in FIG. 3) which are, in turn, electrically coupled to internal conductive wires (not shown in FIG. 3) of lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may comprise a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors.

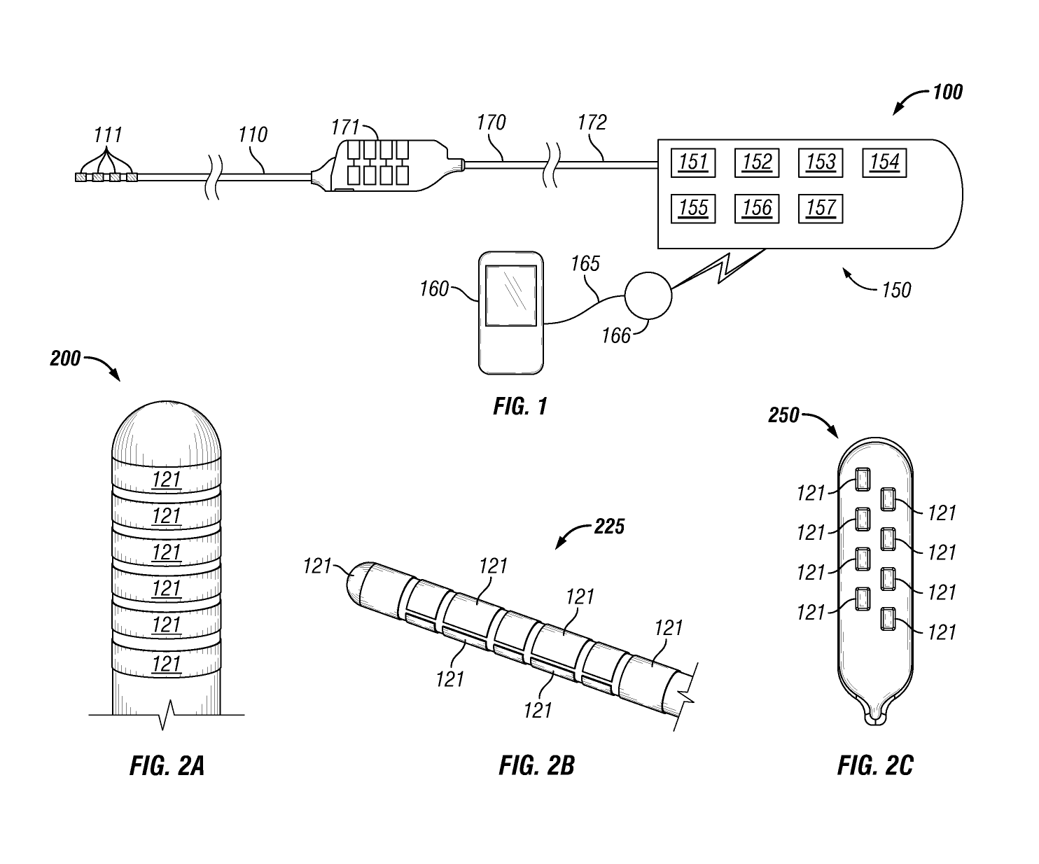

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Patent Publication No. 2010072657, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

Although not required for all embodiments, the lead bodies of lead(s) 110 and extension component 170 may be fabricated to flex and elongate in response to patient movements upon implantation within the patient. By fabricating lead bodies according to some embodiments in this manner, a lead body or a portion thereof is capable of elastic elongation under relatively low stretching forces. Also, after removal of the stretching force, the lead body is capable of resuming its original length and profile. For example, the lead body may stretch 10%, 20%, 25%, 35%, or even up or above to 50% at forces of about 0.5, 1.0, and/or 2.0 pounds of stretching force.

The ability to elongate at relatively low forces may present one or more advantages for implantation in a patient. For example, as a patient changes posture (e.g., “bends” the patient's back), the distance from the implanted pulse generator to the stimulation target location changes. The lead body may elongate in response to such changes in posture without damaging the conductors of the lead body or disconnecting from pulse generator. Also, the ability to “steer” the lead while implanting the lead using a suitable steering stylet may be improved utilizing such a compliant lead according to some embodiments. Fabrication techniques and material characteristics for “body compliant” leads are disclosed in greater detail in U.S. Patent Publication No. 20070282411, entitled “Lead Body Manufacturing,” filed Mar. 31, 2006, which is incorporated herein by reference.

Controller device 160 may be implemented to recharge battery 154 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery 154 by charging circuitry 156. Charging circuitry 156 may also communicate status messages to controller 160 during charging operations using pulse-loading or any other suitable technique. For example, controller 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

Example commercially available neurostimulation systems include the EON MINI™ pulse generator and RAPID PROGRAMMER™ device from St. Jude Medical, Inc. (Plano, Tex.). Example commercially available stimulation leads include the QUATTRODE™, OCTRODE™, AXXESS™ LAMITRODE™, TRIPOLE™, EXCLAIM™, and PENTA™ stimulation leads from St. Jude Medical, Inc. Other commercially available systems and leads include the PRIMEADVANCED™ and RESTORE™ neurostimulators and PISCES™ and SPECIFY™ leads available from Medtronic, Inc. (Minneapolis, Minn.). Other systems and leads include the PRECISION™ neurostimulation system and related stimulation leads from Boston Scientific Neuromodulation Corp. (Valencia, Calif.).

In some embodiments, one or more stimulation leads are placed within the epidural space of the patient. One or more other stimulation leads are placed in subcutaneous tissue.

According to representative embodiments, system 100 is preferably employed to treat chronic pain in a patient. The chronic pain may involve intractable back pain. The back pain may be a result of spinal stenosis or failed back surgery syndrome (FBSS) as examples. In other embodiments, the chronic pain may be axial neck and shoulder pain, chest wall pain, abdominal wall pain, visceral pain, inguinal or hernia pain (see for example, Mironer, Y. E. and Monroe, T. R. (2012), Spinal-Peripheral Neurostimulation (SPN) for Bilateral Postherniorrhaphy Pain: A Case Report. Neuromodulation: Technology at the Neural Interface. doi: 10.1111/j.1525-1403.2012.00495.x, which is incorporated herein by reference), or similar pain disorders of the trunk of the patient. One or more stimulation leads 110 of system 100 are preferably implanted in the patient with electrodes disposed within the epidural space of the patient at a vertebral level appropriate for the pain being treated. One or more stimulation leads 110 are placed in subcutaneous tissue with electrodes disposed in an area of worst pain.

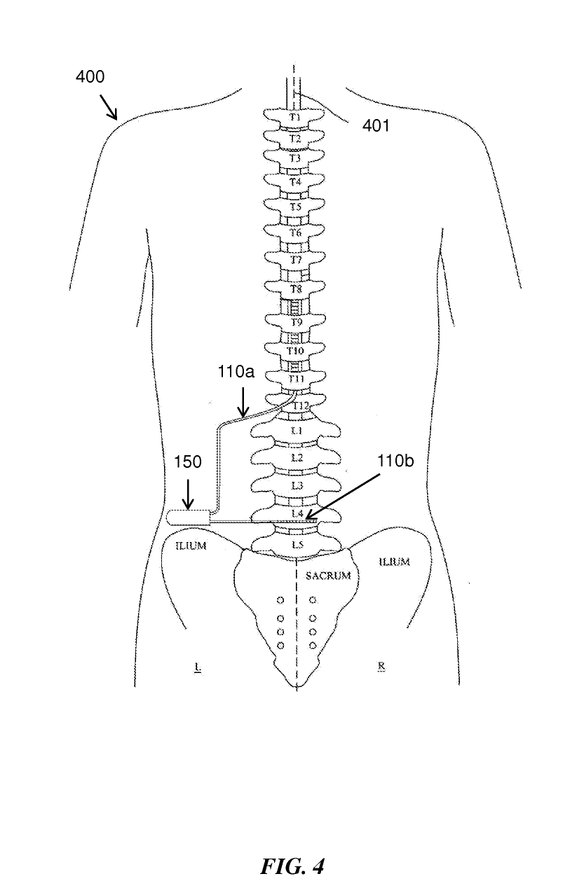

FIG. 3 depicts a fluoroscopy image of leads 110a and 110b implanted to treat chronic pain in a patient according to representative embodiments. FIG. 4 depicts a graphical illustration of leads 110a and 110b implanted according to representative embodiments. Lead 110a is implanted in the epidural space to deliver spinal cord stimulation (SCS) to the patient 400 (as shown in FIG. 4). Lead 110a may generally be placed from T11-12 to T8-T9 (typically at T10) for back pain. Other lead positions may be selected depending upon the nature of the chronic pain in the patient. For example, higher positions may be selected for chest and abdominal pain. In chest wall pain, the epidural lead may be positioned higher and the subcutaneous lead may be positioned parallel to the ribs. Also, for axial neck and shoulder pain, the leads may be implanted in cervical areas. Also, electrodes of lead 110b are positioned over the spine of the patient as shown in FIG. 3. In the embodiment shown in FIG. 3, lead 110a is a percutaneous lead with eight electrodes (e.g., the OCTRODE™ lead available from St. Jude Medical, Inc.), although any suitable stimulation lead (e.g., a surgical paddle-style lead) may be selected according to other embodiments. Lead 110a may be implanted across the midline using the known “midline anchoring” technique to minimize possible migration of lead 110a (see, A NEW TECHNIQUE OF “MIDLINE ANCHORING” IN SPINAL CORD STIMULATION DRAMATICALLY REDUCES LEAD MIGRATION, Neuromodulation 2004:7:32-37 by Mironer Y E, Brown, C, Satterthwaite J R et al. Although lead 110a is shown to be positioned for spinal cord stimulation, other embodiments may deliver electrical stimulation to other neural tissue using a lead implanted within the epidural space. For example, a paddle lead may be implanted to one side of the epidural space to deliver electrical stimulation to nerve roots (and to conduct “cross-talk” stimulation as discussed herein). Such an implant technique may be appropriate for patients having prior spinal fusion procedures where spinal cord stimulation is not available at the appropriate vertebral level.

Lead 110b is implanted in subcutaneous tissue for peripheral nerve field stimulation (PNFS). A percutaneous lead similar to the type of lead selected for lead 110a may be selected for lead 110b. Alternatively, lead 110b may be especially adapted for PNFS and include one or more anchoring elements to retain lead 110b in a desired position within subcutaneous tissue. Lead 110b is placed such that one or more electrodes of lead 110b are employed to stimulate nerve fibers in or adjacent to the implant location. The distal end of lead 110b (with its electrodes) are positioned in a manner that is substantially perpendicular to the spinal axis 401 as shown in FIG. 4. Electrodes of lead 110b are preferably positioned in an area corresponding to the worst pain of the patient. A suitable location for axial back pain may typically be found between L2-L3 and L5-S1 (frequently at L4-5).

As shown in FIG. 4, it may be advantageous during the implant procedure to employ the subcutaneous IPG pocket for implantation of the subcutaneous lead 110b. Specifically, it is possible to advance the tunneling tool directly to or from the IPG pocket for providing subcutaneous access to the PNFS site thereby reducing the number of incisions experienced by the patient.

Multiple stimulation programs may be provided for stimulation of the patient using leads 110a and 110b. For example, in one program (“Program 1”), the epidural stimulation and PNFS occur independently using respective bipolar configurations of active electrodes on leads 110a and 110b. The bipolar configurations generally tend to limit the resulting current flow to tissue immediately adjacent to the active electrodes. In another program (“Program 2”), one or more electrodes of epidural lead 110a may be programmed to function as anodes with one or more electrodes of PNFS lead 110b functioning as cathodes. In yet another program (“Program 3”), one or more electrodes of epidural lead 110a may be programmed to function as cathodes with one or more electrodes of PNFS lead 110b functioning as anodes. In Programs 2 and 3, the electrodes on one given lead 110 are selected to function in a monopolar manner. That is, all active electrodes on one respective lead 110a are set to the same polarity. Also, all electrodes on PNFS lead 110b may preferably (but necessarily) be set to an active state. In Program 3, a subset of electrodes of epidural lead 110a may be programmed to be active. As known in the art, a patient controller device may be employed by the patient to select from these various programs as deemed suitable by the patient. Also, the various programs may be selected for the patient's therapy according a scheduling or cycling protocol.

It has been observed that a patent receiving stimulation from system 100 according to Program 1 will typically feel PNFS covering a small area of the patient's back. With Program 2, a patient will often experience a larger area of the back covered by the stimulation with or without some stimulation in one or both legs. With Program 3, a patient may be expected to experience relatively wide axial back coverage. With Program 3, some patients have been observed to experience stimulation in abdominal and/or flank areas. Although the abdominal and flank coverage has not been typically reported as uncomfortable or painful, the abdominal and/or flank coverage may be eliminated by changing the active electrodes on epidural lead 110a in Program 3.

In addition to the selection of the electrode states for the various Programs 1-3, any other suitable stimulation parameters may be selected. In some representative embodiments, for stimulation involving the PNFS electrodes of lead 110b, frequencies between 10 Hz to 80 Hz may be employed according to some representative embodiments. Pulse widths for constant current pulses may range from 250 μsec to 400 μsec with pulse amplitudes ranging from 4 mA to 11 mA.

In applying stimulation to patients according to some representative embodiments, “cross-talk” occurs between electrodes of the epidural lead 110a and electrodes of the PNFS lead 110b. To obtain “cross-talk,” one or more electrodes of the epidural lead 110a may be selected to function as cathodes with one or more electrodes of PNFS lead 110b simultaneously selected to function as anodes (or vice versa). It is believed that active electrodes of opposite polarities on the respective leads 110a and 110b causes current flow between the epidural site and the PNFS site. It is believed that the current may follow the path of highest conductivity (possibly through nerve roots). The observation of abdominal wall coverage supports the conclusion that nerve root stimulation may occur as a result of such cross-talk. Notwithstanding theoretic considerations, it has been observed that “cross-talk” programs for system 100 are rather effective in providing coverage for axial back pain. Also, it has been observed that patients often prefer low frequencies for such stimulation (e.g., at or below 20 Hz). Further, the pulse width and current parameters (e.g., less than 300 μsec and less than 5 mA) are often selected that are lower than parameters typically employed for PNFS stimulation alone.

Cross-talk stimulation with epidural and PNFS according to some embodiments has been provided to patients in multiple groups. In one group of twenty (20) patients (70% female and 30% male), stimulation was provided using epidural and PNFS using the aforementioned Programs 1-3. Eighteen (18) out of the twenty patients experienced more than 50% pain relief through trial stimulation thereby representing a 90% success rate.

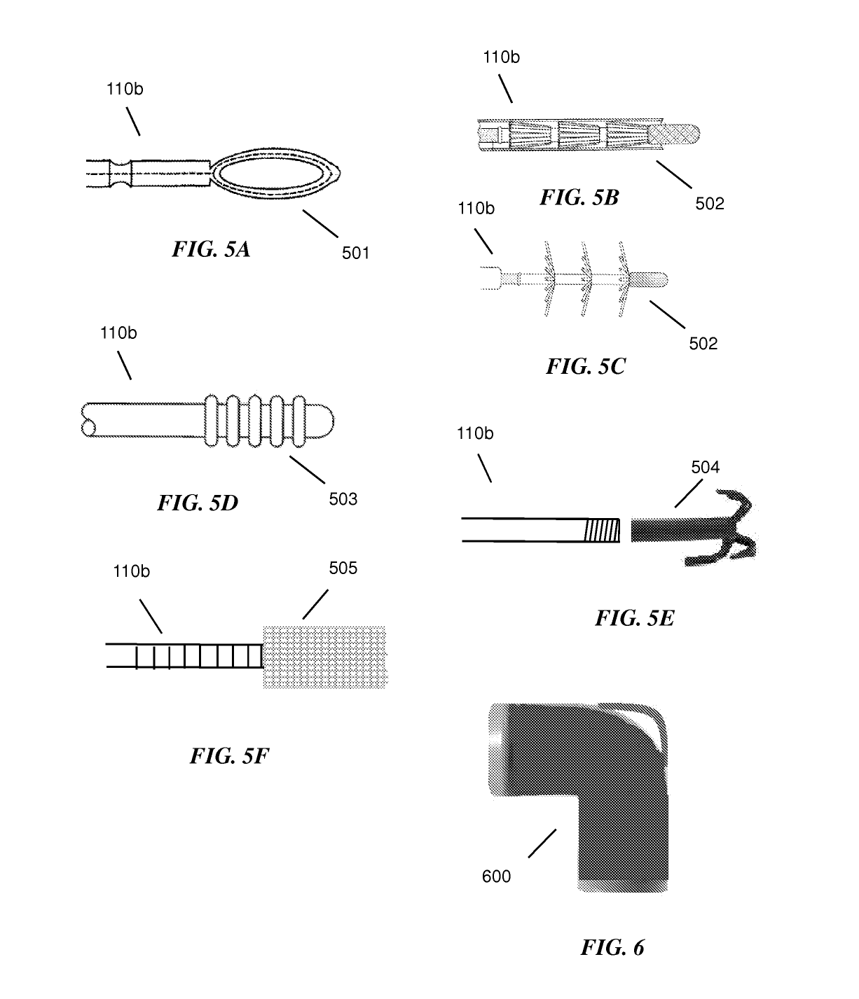

In some embodiments, lead 110b is adapted to minimize migration to maintain electrodes of lead 110b at a desired location relative to the patient's spine. FIGS. 5A-5F depict respective mechanisms for retaining a PNFS lead at a desired location. FIG. 5A depicts lead 110b having suture loop 501 disposed at the very distal end of lead 110b. Suture loop 501 may be sutured to soft tissue of the patient to retain lead 110b at its desired location. FIGS. 5B and 5C depict lead 110b with expandable tines 502. The tines may be held in a retracted state (e.g., within a sheath) as shown in FIG. 5B and expanded (as shown in FIG. 5C) after lead 110b is placed in the appropriate location. FIG. 5D depicts lead 110b with grooves for fixating lead 110b in place with sutures.

In some embodiments, the retention structures may be placed onto the distal end of lead 110b after lead 110b is positioned at the implant site. FIG. 5E depicts retention structure 504 that is adapted to mate with the distal end of lead 110b (e.g., using various mating structures including flanges, grooves and threads, etc.). Alternatively, a biocompatible adhesive may be employed to secure retention structure 504 to lead 110b. By permitting, the retention structure to be placed on lead 110b after positioning at the implant site, the retention structure may possess a size and configuration that is not easily passed through a tunneling tool. Such a structure may be beneficial in retaining the PNFS lead 110b at its desired location. FIG. 5F depicts polypropylene mesh 505 that may be placed over the distal end of lead 110b. Tissue in-growth may occur through mesh 505 thereby retaining the distal end of lead 110b in the implant location.

FIG. 6 depicts silicone anchor implant device 600 for securing a stimulation lead within a patient between the epidural space and the subcutaneous pocket created for the implantable pulse generator. Anchor implant device 600 may comprise internal multiple lumens (not shown) for securing multiple stimulation leads. Anchor implant device 600 may be sutured into tissue at a desired location relative to the subcutaneous pocket for the implantable pulse generator.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. A method of treating chronic pain in a patient, the method comprising:
placing a first stimulation lead into the epidural space of the patient;
placing a second stimulation lead in subcutaneous tissue with electrodes of the second stimulation lead disposed in a configuration that is substantially perpendicular to an axis defined by the spine of the patient and in an area of back pain of the patient;
generating electrical pulses for application to tissue of the patient; and
applying the electrical pulses to the tissue of the patient simultaneously using electrodes of the first stimulation lead and electrodes of the second stimulation lead, wherein active electrodes on the first stimulation lead are set to a first polarity and active electrodes on the second stimulation lead are set to a second polarity that is opposite to the first polarity while the applying is performed.

2. The method of claim 1 wherein the active electrodes of the first stimulation lead are set as cathodes and the active electrodes of the second stimulation lead are set as anodes.

3. The method of claim 1 wherein the active electrodes of the first stimulation lead are set as anodes and the active electrodes of the second stimulation lead are set as cathodes.

4. The method of claim 1 wherein a pulse frequency for the generating is selected to be 20 Hz or less.

5. The method of claim 1 further comprising:
determining whether the patient experiences abdominal wall stimulation; and
adjusting active electrodes of the first stimulation lead in response to the determining.

6. The method of claim 1 further comprising:
changing polarities of the active electrodes on the first and second stimulation leads to modify stimulation coverage over the patient's lower back.

7. The method of claim 1 wherein the chronic pain is a result of spinal stenosis.

8. The method of claim 1 wherein the chronic pain is a result of failed back surgery syndrome.

9. The method of claim 1 wherein the placing the second stimulation lead comprises:
tunneling through a subcutaneous pocket created to retain an implantable pulse generator to the area of back pain to create a path for the second stimulation lead.

10. The method of claim 1 further comprising:
attaching a retention structure to a distal end of the second stimulation lead after tunneling the second stimulation lead to the area of back pain.
